(19) United States
(12) Patent Application Publication
(10) Pub. No.: US 2004/0023928 A1
(43) Pub. Date: Feb. 5, 2004

(54) Phosphonate nucleotide and thiadiazole compounds for the treatment of smallpox

(76) Inventor: Joseph Matthew Colacino, Indianapolis, IN (US)

Correspondence Address: ELI LILLY AND COMPANY, PATENT DIVISION, P.O. BOX 6288, INDIANAPOLIS, IN, 46206-6288, US

(21) Appl. No.: 10/273,809

(22) Filed: Oct. 17, 2002

Related U.S. Application Data
(60) Provisional application No. 60/335,189, filed on Oct. 31, 2001.

(51) Int. Cl. A61K 31/675; A61K 31/433

(52) U.S. Cl. 514/81; 514/363

(57) ABSTRACT

The present invention provides methods of treating smallpox and other poxvirus infections with a pharmaceutically effective amount of LY582563 or LY217896.

### BACKGROUND OF THE INVENTION

It has been known for some time that antiviral drugs can be found among the general families of phosphonate nucleotides and thiadiazoles.

### BRIEF SUMMARY OF THE INVENTION

The present invention provides methods of treating smallpox and other poxvirus infections with a pharmaceutically effective amount of LY582563 or LY217896.

### DETAILED DESCRIPTION OF THE INVENTION

As used herein:

The term “LY582563” refers to the anti-viral compound 2-amino-9-[2-[bis(2,2,2-trifluoroethyl)phosphonylmethoxy]ethyl]-6-p-methoxyphenylthiopurine. This phosphonate nucleotide is described in U.S. Pat. No. 5,840,716 (see Example 3; Compound 4), incorporated herein by reference.

The term “LY217896” refers to the anti-viral compound 1,3,4-thiadiazole-2-cyanamide. This thiadiazole is described in U.S. Pat. No. 4,835,168 (see Example 6), incorporated herein by reference.

At present, with the increased risk of bio-terrorism after the terrible events of Sep. 11, 2001, there is a need for the treatment of various viral scourges, like small pox for which there is currently no effective treatment. The present invention provides a method of treating the disease caused by the small pox virus by administering LY582563 or LY217896.

### EXAMPLE 1

### 2-amino-9-[2-[bis(2,2,2-trifluoroethyl)-phosphonylmethoxy]ethyl]-6-p-methoxyphenylthiopurine

2-Chloroethyl chloromethyl ether (87 g, 670 mmol) and Tris(2,2,2-trifluoroethyl)phosphite (200 g, 610 mmol) are heated at 160° C. for 7 hours to obtain 2-[bis(2,2,2-trifluoroethyl)phosphonylmethoxy]ethyl chloride quantitatively.

2-[Bis(2,2,2-trifluoroethyl)phosphonylmethoxy]ethyl chloride (206 g) is dissolved in methyl ethyl ketone (2,000 ml) and the solution is heated under reflux with sodium iodide (270 g) for 8 hours. After completion of the reaction, the mixture is cooled to room temperature and concentrated to dryness. The residue is dissolved in chloroform/hexane and adsorbed on a silica gel column, and then eluted with chloroform/hexane to give 2-[bis(2,2,2-trifluoroethyl)-phosphonylmethoxy]ethyl iodide quantitatively.

2-Amino-6-chloropurine (15.0 g, 88 mmol) is suspended in dimethylformamide (360 ml) and treated with 1,8-diazabicyclo[5.4.0]-undec-7-ene (13.9 ml, 93 mmol) at 80° C. for 1 hour. Then, 2-[bis(2,2,2trifluoroethyl) phosphonylmethoxy]ethyl iodide (23.8 ml) is added to the reaction mixture and subjected to a reaction at 100° C. for 5 hours. After completion of the reaction, the mixture is cooled to room temperature and concentrated to dryness. The residue is dissolved in chloroform and adsorbed on a silica gel column, and then eluted with 5%-methanol in chloroform to give 2-amino-9-[2-[bis(2,2,2-trifluoroethyl) phosphonylmethoxy]ethyl-6-chloropurine (23.3 g, yield 56%).

Triethylamine (2.1 ml) and p-methoxythiophenol (3.1 ml) are added to a solution of 2-amino-9-[2-[bis(2,2,2-trifluoroethyl)phosphonyl-methoxy]ethyl]-6-chloropurine (7.1 g) in dimethylformamide (68 ml) and the mixture is stirred at 100° C. for 2 hours. The reaction mixture is cooled to room temperature and concentrated to dryness. The residue is dissolved in chloroform and adsorbed on a silica gel column, and then eluted with 5%-methanol in chloroform to give 2-amino-9-[2-[bis(2,2,2-trifluoroethyl)-phosphonylmethoxy]ethyl]-6-p-methoxyphenylthiopurine (5.0 g, yield 61%).

m.p.: 93-95° C. (diisopropyl ether)

¹H-NMR(CDCl₃, δ) : 3.85(s,3H) , 3.92-4.00(m,4H) , 4.24-4.45(m,6H), 4.75(s,2H), 6.95(d,J=9.0 Hz,2H), 7.53(d,J=9.0 Hz,2H), 7.71(s,1H)

### EXAMPLE 2

### 1,3,4-thiadiazole-cyanamide

A mixture of 4.8 g of 1,3,4-thiadiazol-2-ylthiourea in 45 ml of 1N sodium hydroxide, 15 ml of ethanol and 4 ml of methyl iodide is heated at 40° C. for ten minutes. The mixture is acidified by adding 50 ml of 1N hydrochloric acid. The reaction mixture is concentrated by evaporation under reduced pressure. The precipitated solid is collected by filtration and dried to give 3.02 g of N¹-1,3,4-thiadiazol-2-ylcarbamimidothioic acid, methyl ester.

A solution of 3.48 g (20 mM) of N¹-1,3,4-thiadiazol-2-ylcarbamimidothioic acid, methyl ester in 200 ml of dichloromethane containing 4 g (20 mM equivalents based on 85% purity) of meta-chloroperbenzoic acid is stirred at 24° C. for two hours. The mixture is filtered and the precipitate is then stirred with 40 ml of water for two hours. The solid is collected by filtration and dried to give 1.7 g of 1,3,4-thiadiazole-2-cyanamide.

FD mass spec.: 126

Analysis Calc. for C.sub.3 H.sub.2 N.sub.4 S:

Theory: C, 28.57; H, 1.60; N, 44.42; S, 25.42.

Found: C, 28.95; H, 1.80; N, 43.60; S, 24.87.

The compounds of the present method are preferably administered as a pharmaceutical formulation. Therefore, as yet another embodiment of the present invention, a pharmaceutical formulation useful for treating various diseases including smallpox in mammals is provided comprising LY217896 and/or LY582563 with a pharmaceutical carrier, diluent or excipient therefor.

In the normal practice of pharmaceuticals, the active compound to be employed in the present invention is preferably formulated with one or more adjuvants, carriers, and/or diluents. The identity of suitable such components is well known to those skilled in the art, as is the method of mixing such components. For oral administration, the subject compound can be formulated as a capsule, tablet, suspension, or other form for oral delivery. For intravenous infusion, the compound can be dissolved in a suitable intravenous fluid, such as physiological saline, 5% dextrose solution, or the like.

The compounds are effectively administered orally, topically or parenterally. Oral administration is a preferred administration route for a medicament of the present invention. While the particular dose to be administered will be determined by the precise viral infection to be treated or guarded against and its severity, the route of administration, and related circumstances will be determined by attending medical practitioners.

LY217896 is active over a wide range of dose levels. A therapeutically effective dose will range from about 0.1 to about 100 mg/kg, and more typically about 0.5 to about 25 mg/kg.

LY582563 is also active over a wide range of dose levels. The therapeutically effective dose may generally be, for use of an oral administration, 0.1 to 500 mg/kg, preferably 1 to 50 mg/kg per day for an adult. For administration by injection, a therapeutically effective dose range may be from about 0.01 to about 50 mg/kg, preferably 0.1 to 5 mg/kg per day for an adult.

The dose may be appropriately increased of decreased depending on age, conditions, symptoms, or the presence of a co-administered drug.

We claim:

1. A method of treating smallpox and other poxviruses in a mammalian patient in need thereof comprising administering a therapeutically effective dose of the compound 1,3,4-thiadiazole-cyanamide or a pharmaceutically acceptable salt or solvate thereof.

2. A method of treating smallpox and other poxviruses in a mammalian patient in need thereof comprising administering a therapeutically effective dose of the compound 2-amino-9-[2-[bis(2,2,2-trifluoroethyl)-phosphonylmethoxy]ethyl]-6-p-methoxyphenylthiopurine.
